(19) United States
(12) Patent Application Publication
(10) Pub. No.: US 2005/0228538 A1
(43) Pub. Date: Oct. 13, 2005

(54) Methods and apparatus including an improved user interface for providing custom hair coloring products to a user

(76) Inventor: Christian Limburger, Norwalk, CT (US)

Correspondence Address: JEFFREY L. BRANDT;c/o PORTFOLIO IP, P.O. BOX 52050, MINNEAPOLIS, MN, 55402, US

(21) Appl. No.: 10/821,011

(22) Filed: Apr. 8, 2004

(51) Int. Cl. G06F 17/00; A61K 7/13

(52) U.S. Cl. 700/239

(57) ABSTRACT

There is provided methods and systems enabling untrained customers to operate a hair colorant mixing machine. Graphical interfaces are provided to the customer enabling her to identify her existing hair color and to select a desired change to that hair color. Responsive to the existing hair color and the desired change, using packaged dyes and chemicals, the machine formulates a custom hair colorant, mixes that colorant and packages and dispenses that colorant to the customer.

### RELATED APPLICATIONS

The present application is related to co-pending U.S. patent application Ser. No. ______, by Limburger, C., et al., titled: Apparatus and Methods for Selecting, Formulating, Mixing & Dispensing Custom Hair Coloring Products for a User, filed on same date herewith.

### FIELD OF THE INVENTION

The present invention relates generally to the field of hair coloring and more particularly to apparatus and methods for selecting and dispensing custom hair coloring products.

### BACKGROUND OF THE INVENTION

Countless individuals all over the world choose to change their appearance through the use of hair coloring agents, typically in the form of pre-packaged dye products. One example of pre-packaged dye products are those products commercially available for purchase by a consumer in retail stores, such as pharmacies, hair salons and other locations. Available from a multitude of suppliers such as Clairol®, Revlon®, L'Oreal® and others well known to the reader, these products include pictures and charts supporting customers in selecting the correct product to provide the desired results. Because pictures and writings can communicate a limited amount of information, and because each individual has different hair characteristics that affect the coloring process, standard products provide a very ‘hit-or-miss’ result in yielding the desired hair color.

Various efforts have been made to simplify the selection of hair-coloring formulations. For example, computerized systems and displays have been used to collect data from customers, suggest standard product selections and generate electronically-displayed images of anticipated hair coloring results. See, for example, U.S. Pat. No. 4,434,467, titled: Hair Coloring Calculator, to Scott. See also U.S. published patent application 2002/0010556, titled: Method for Analyzing Hair and Predicting Achievable Hair Dyeing Ending Colors, to Marapane et al.

In some instances it has been the practice to use cameras or other sensors to take and display images of a customer as part of the process of selecting a hair coloring product. See, for example, WO03020072, titled: Method for a Hair Colour Consultation, by Werner, et al. See also United States published patent application 2003/0065450 wherein a computer interface and an image acquisition device are both used to collect information about a customer and to dispense apparently pre-packaged hair coloring products. While the collection of additional data electronically and through sensors may improve the results of the hair coloring process, these apparatus and processes still suffer significant drawbacks. One major drawback is the ability to select from a relatively small number of standard commercial products in order to achieve the specific result desired by each individual customer.

One interesting invention is shown in German application 101 14 060 A1. This application teaches a machine for custom mixing of a coloring product, from dyes and the appropriate other formulating chemicals, for each individual customer of a salon. Upon evaluating a customer's hair, a trained operator can control the machine to mix a customized coloring product likely to meet each individual customer's needs. The machine suffers, however, from several disadvantages. A first disadvantage is that as disclosed the device enables only the production of classic, ammonia-containing hair dyes, in which only the peroxide needs to be added prior to use. Another disadvantage is that of requiring operation by a highly experienced and trained person. In operation, the exact color(s) and quantity(s) of dyes must be entered for each desired hair color product. Knowing how to select such dyes requires training well beyond the skills and abilities of a casual user and certainly well beyond the capabilities of a customer.

The present inventors have determined that there exists a need in the art for machines and processes which enable an ordinary customer, untrained in the operation of special equipment or the process of hair dye mixing, to formulate a hair coloring product especially for her hair. Such an innovation would desirably share the beneficial characteristics of pre-formulated products, including immediate availability and cost-effectiveness, along with the beneficial characteristics of custom-mixed products, including the increased likelihood that the product will provide the desired result for the customer.

### SUMMARY OF THE INVENTION

There are provided herein methods and systems which, through the use of simple user interfaces, enable a customer to obtain a custom-mixed hair colorant. The invention provides for the use of simple interfaces including the display of hair colors enabling the customer to accurately and easily identify both an existing hair color and a desired change to their existing hair color. The invention then provides for the selection and mixing of appropriate dyes and other chemical formulants to provide the custom-mixed hair colorant. The colorant may be packaged and dispensed directly to the customer.

In accordance with one embodiment of the invention there are provided methods and apparatus for formulating a hair colorant, an apparatus comprising: a plurality of dyes; means for receiving from a customer an indication of an existing hair color; means for visually displaying to the customer a plurality of optional changes to the existing hair color; means for receiving from the customer, responsive to the means for visually displaying, an indication of a desired change to the existing hair color; and means for selecting from the plurality of dyes at least one selected dye for mixing into a hair colorant.

In accordance with another embodiment of the invention there are provided methods and systems for formulating a hair colorant, a system comprising: a processor; a touch-screen display connected to the processor; a memory connected to the processor and storing instructions to control the processor to perform the steps of: display on the touch-screen display a plurality of hair colors; receive from a customer through the touch-screen display an indication of an existing hair color; display to the customer on the touch-screen display a plurality of optional changes to the existing hair color; receive from the customer on the touch-screen display an indication of a desired change to the existing hair color; and select, from a plurality of dyes, at least one selected dye for mixing into a hair colorant.

The present invention provides significant advantages over the prior art. Because it can be operated by an unskilled customer, it may be used in retail stores and other locations where there is customer demand but no skilled hair colorist. Because each color formulation is custom-mixed from a limited number of ingredients, many different colorant products may be formulated using a relatively small space, for example a relatively small area in a retail store. Further, because each hair colorant is custom-formulated in accordance with accurately provided customer instructions, the likelihood of a customer achieving a desired result is significantly greater than with off-the-shelf products.

### DESCRIPTION OF THE DRAWING FIGURES

These and other objects, features and advantages of the invention will become apparent through a consideration of the following detailed description of the invention when considered in conjunction with the drawing Figures, in which:

FIG. 1 is a block diagram of a hair colorant mixing and dispensing machine in accordance with the present invention;

FIG. 2 is a flow chart showing an overview of a process for selecting, mixing and dispensing a hair colorant in accordance with the present invention;

FIG. 3 is a flow chart showing the process for determining a user's existing hair color;

FIG. 4 is a flow chart showing a process for determining a user's desired change to their hair color;

FIG. 5 is a flow chart showing a process for mixing and dispensing a new hair colorant in accordance with the present invention;

FIG. 6 illustrates a first graphical user interface displayable on a device such as a computer screen in support of the present invention;

FIG. 7 illustrates a second graphical user interface displayable on a device such as a computer screen in support of the present invention; and

FIG. 8 is a flow chart showing a process for selecting dyes to formulate a hair colorant in accordance with the present invention.

### DETAILED DESCRIPTION OF THE INVENTION

The invention is relative to a method for producing hair dyes and/or intensive toners (demi-permanent, semi-permanent hair dyes) in which the intermediates (developer and coupler) and the direct dyes are dissolved in liquid or creamy carrier masses that are mixed together by a computer-controlled device based on recipes stored in the computer to a tone mixture on site, e.g., in a hairdressing salon. As noted above, the invention provides significantly simplified and useful user interfaces enabling unskilled customers to operate and product the desired colorants.

As noted herein above, German application 101 14 060 A1, incorporated herein by reference in its entirety, teaches a method and a device with which, in its previously disclosed form, only the production of classic, ammonia-containing hair dyes is possible. In accordance with the present invention, classic oxidation hair dyes and also intensive toners (semi-permanent) can be produced given appropriate equipping of the storage device with base components and appropriate recipes. The intermediates (developer and coupler), required for creating the tone, and the direct dyes are dissolved individually here together with other chemicals in a carrier mass and filled as so-called base components into flexible bags. The bags are placed in magazines fastened, e.g., on a turntable, and connected via connectors to pumps (e.g., piston pumps). The tone is composed via a computer in which the recipes and/or the composition of the desired tone by mixing the individual base components are stored, by fetching the recipe for the particular hair color desired. This type of ready-to-use production of hair dyes and/or intensive toners has the advantage that many tones can be produced on site, that is, e.g., in a hairdressing salon, with few base components. As is described in German application 101 14 060 A 1, the base components filled in the bags yield the ready-to-use hair dye after the mixing process. Only the peroxide needs to be added prior to use. Of course, such a device can also be used to produce physical toners to the extent that other, appropriately designed base components are added.

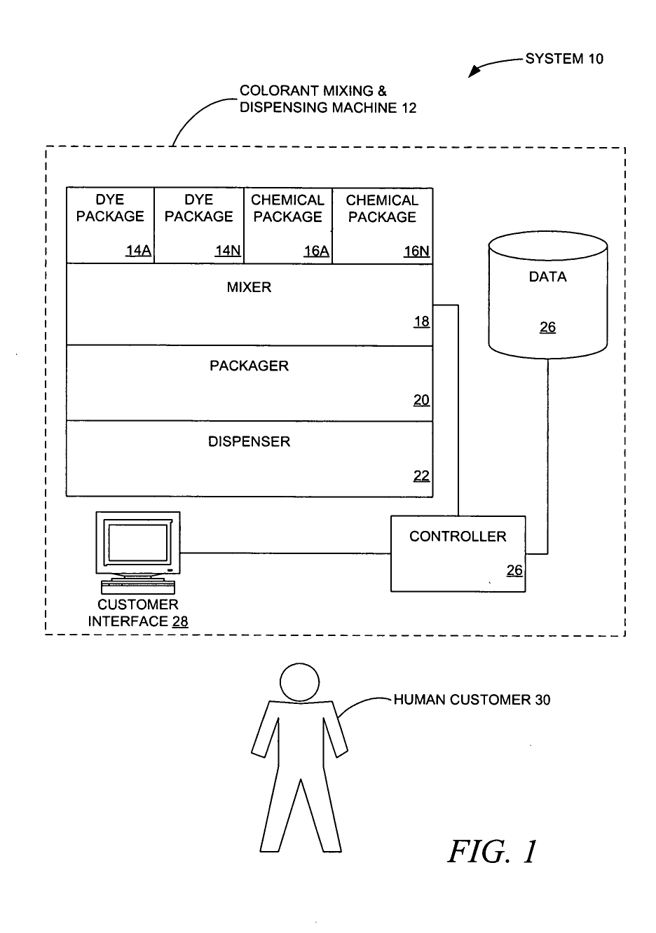

With reference now to FIG. 1, a block diagram of a system 10 is shown including a hair colorant mixing and dispensing device or machine 12, for example of the type shown and described in German application 101 14 060 A 1, but with the inclusion of a user interface in accordance with one aspect of the invention as shown and described herein below.

As shown in FIG. 1, machine 12 includes an assortment of packaged dyes, indicated as dye bags 14A-N, along with various bags and containers of other appropriate and necessary chemicals, indicated as chemical packages 16A-N. While for purposes of illustration two packages of dye and two packages of other chemicals are shown, it will be understood these packages are relatively small and there can be many packages of each included in machine 12. The exact chemistry of the dye formulation process is described in further detail herein below. Various mechanical devices are shown for mixing 18 the dyes and chemicals to form a hair colorant, packaging 20 and dispensing 22 the packaged hair colorant. Again, numerous such devices will be known to the reader.

As used herein, the terms “dye” and “colorant” are used interchangeably to described a mixed formulation to be applied directly to the hair. The term “dye” is also used in context to describe an unmixed dye material.

Continuing with reference to FIG. 1, machine 12 further includes a computer controller 26 connected to a data storage device 24. Controller 26 can comprise one of many well-known computers or processors programmed to operate machine 12 in accordance with the processes described herein below. Storage device 24 comprises a conventional and appropriate combination of magnetic, optical and semiconductor memory appropriate to store the data and process control software described herein below. In accordance with the invention, machine 12 further includes a user, or customer interface 28 connected to controller 26 for operation by a human operator 30, typically a customer desiring a hair colorant but not trained in the mixing or formulation of hair colorant dyes and chemicals. User interface 28 comprises a terminal, for example incorporating a touch screen, capable of displaying color pictures to user 30 and receiving instructions there from in the manner described below.

Considering now the chemistry of formulating hair colorants, as is known in the art, oxidation hair dyes are generally composed of intermediates (developers and couplers) and direct dyes dissolved in a stable manner in an ammonia-containing carrier mass.

The carrier mass used can be present in liquid or creamy form such as described, e.g., in the “Handbuch der Kosmetika und Riechstoffe” [Handbook of Cosmetics and Perfumes] published by the A. Hütig Verlag, Heidelberg, 2^{nd} edition, volume 3, or in “Grundlagen für klare, flüssige Haarfarben” [Bases for Clear, Liquid Hair Dyes] by Hugo Janistin. Other examples can also be found in “The Chemistry and Manufacture of Cosmetics” by Maison G. de Navarre, volume IV, published by Allured Publishing Corp., Illinois, USA, 3d edition, or in “Grundlagen und Rezepturen der Kosmetik” [Bases and Recipes of Cosmetics] by Karlheinz Schrader published by the A. Hütig Verlag, Heidelberg, 2^{nd} edition.

The appropriate intermediates (developer and coupler) in direct dyes as well as one or more alkylizing agents are added to these carrier masses or to the particular carrier mass selected. The finished product produced in this manner, the oxidation hair dye, is subsequently filled into containers such as tubes or bottles. The oxidation hair dye is mixed with 1 to 3 parts of a peroxide solution immediately before use and applied onto the hair to be dyed.

Classic oxidation hair dyes contain ammonia in order to simultaneously achieve a certain brightening effect of the natural hair during the dying process; on the other hand, intensive toners contain only a little or no ammonia, but contain other alkalizing agents such as sodium hydroxide, ethanolamine, etc. If the hairdresser wants to offer both types of oxidation dyes he must therefore have a double assortment.

In accordance with one embodiment of this invention, the ability to selectively product classic hair dyes or intensive toners on site with the same base components is provided in that the intermediates used (developer and coupler) and the direct dyes are dissolved in a precursor stage in a carrier mass with only as much alkalizing agent being added as is necessary for the dissolving and stabilizing of the intermediates (developer and coupler), of the direct dyes and of the carrier mass (called base components for short), and in a further step after the mixing together of the individual base components to a tone in a container, ammonia (for producing classic oxidation hair dyes) or substitutes (for producing intensive toners) is/are selectively added.

As a result of this method only as much alkalizing agent (e.g., sodium hydroxide) is added to the base components in a precursor stage as is necessary for the dissolving and the stabilizing of the intermediates (developer and coupler), the direct dyes and the carrier mass. For completion, other alkalizing agents such as ammonia and/or ethanolamine and/or sodium hydroxide must be added and worked into the carrier mass in a further stage depending on the desired type of coloring (classic hair dyes or intensive toners). For dyeing or toning, the particular alkalizing agent desired or required is added to the dye mass immediately before use and the entirety is mixed as customarily in a further step with peroxide and is applied.

The advantage of this procedure is that the hairdresser can produce the classic ammonia-containing oxidation hair dye as well as the ammonia-free intensive toner himself, e.g., immediately before use in the salon, with the same base components by selecting the desired alkalizing agent in the following stage.

Customary alkalizing agents can be used as alkalizing agents in the second stage.

It has proven to be advantageous in the production of the base components to combine the separate aqueous phases and fatty phases at 70 to 80° C. and to mix in the treatment substances and perfume substances at approximately 50° C. The filling of the ready base components into flexible bags takes place in the ideal instance at 45 to 40° C.

In order to be able to carry out the production of the classic hair dye or intensive toner on site, that is, in the salon, a device of the type shown in FIG. 1 has proven to be advantageous in which the bags with the base components can be suspended or placed and which comprises control means for filling the masses into a container. The control of the device is designed in such a manner that at first the base components are combined and subsequently alkalizing agents are added in a further step during which the hairdresser can decide between the various alkalizing agents, depending on the type of dyeing. The peroxide is subsequently added only after the conclusion of the first two stages or, if the hair dye or intensive toner is not to be used in a salon, is placed in a container, e.g., a bottle. The hairdresser has the advantage, due to the possibility of selecting the alkalizing agents, of being able to produce both classic, ammonia-containing hair dyes as well as intensive toners in the salon with the same device and by using the same base components.

In order to be able to meet all requirements such as, e.g., the shading of the tone, the brightness of the tone, etc., the base components, the alkalizing agents and the peroxides are held available in various concentrations in the device.

An exemplary embodiment of the invention is described in detail as follows. The device 12 for producing hair dyes and intensive toners consists as is described in German application 101 14 060 A1 of a motor-driven turntable on which, e.g., magazines or containers for depositing flexible bags such as dye and chemical bags 14A-N and 16A-N are fastened. Each of these bags is connected into mixer 18, e.g., by a connector and a control valve to a pump (e.g., piston pump). The pump exit empties into a dish. A turntable and pumps as well as the control valves in mixer 18 are controlled by a computer program (by software) stored in storage device 24 and operated by controller 26 in such a manner that an amount or a multiple of a given base amount fixed in the recipe is transported by the pumps into the dish. After the amounts fixed by the recipe and corresponding in their composition to the desired tone of the hair dye or intensive toner have been dosed into a dish, the amount in the dish is mixed by mixer 18.

While prior art embodiments of machine 12 have been by necessity placed within salons for operation by trained hairdressers and operators, it will be seen that a significant advantage of the present invention is the ability of the described system 10 to be operated by a customer 30 not trained or skilled in the art of mixing hair colorants. This enables machine 12 to be placed in a variety of non-salon environments, including for example retail stores and outlets where customers typically purchase hair colorants. Machine 12 thus incorporates packaging mechanism 20 for placing the mixed hair colorant into a package, for example a bag, tube or bottle, and dispenser 22 for dispensing the package to customer 30. Again, many such mechanical devices will be readily known to the reader.

Continuing with respect to the chemical formulations of the improved hair colorant mixing process, in one illustrative example and without limitation, 11 magazines with 11 bags are placed on the motor-driven turntable of the device 12 for producing classic hair dyes and intensive toners in accordance with the described method. Of the 11 bags, 7 bags (bags 1 to 7) are filled with base components, 2 bags (bags 8, 9) with alkalizing agent, 1 bag (bag 10) with carrier mass and 1 bag (bag 11) with peroxide. Over 500 color shadings can be produced both as classic ammonia-containing hair dye or as intensive toner with such provisions by means of the device and the entered recipes in as far as the method of the invention is followed, that is, the base components filled into the bags receive only as much alkalizing agent, e.g., sodium hydroxide, in a precursor stage as is necessary for the dissolving and stabilizing of the intermediates (developer and coupler), the direct dyes and the carrier mass. More alkalizing agent, depending on the type of coloring, is added only after the mixing of the base components, that is, after the base components have been placed into a dish or a container in accordance with the recipe. As a result of this two-stage method both ammonia (in the case of the classic hair dye) as well as so-called substitutes such as, e.g., ethanolamine, AMP, etc. (in the case of the intensive toner) can be added. The individual intermediates (developer and coupler), the direct dyes and the associated substances are dissolved in the precursor stage in order to produce the individual base components at 70 to 80° C. in the aqueous phase. The alkalizing agent is subsequently added together with the fatty phase and emulsified. Finally, treatment substances and perfume substances are mixed in during the cooling-off process at approximately 50° C.

When selecting a tone, e.g., by using samples on a color card, the computer can calculate, after the tone has been entered, e.g., in accordance with the prior art by the entry of a code, the individual requirement for the desired color amount based on the recipe in the computer and/or on other selected parameters, and dose this amount into the dish.

This type of production makes a very rapid creating of classic, ammonia-containing hair dyes as well as of intensive toners possible for the hairdresser based on the recipes stored in the memory.

The base components filled into the bags are composed of a liquid, gelatinous or creamy carrier mass with treatment substances, a coupler, a developer, a direct dye (direct dyes) as well as of the amount of an alkalizing agent necessary for dissolution and stabilization.

The following are used as developer:
p-touylenediamine; p-toluylenediamine 0.1 to 20%, p-phenylenediamine 0.1 to 5%, p-aminophenol, 0.1 to 2%, 2-chloro-p-phenylenediamine sulfate 0.1 to 5%, 4-amino-3-methylphenol 0.1 to 5%, N,N-bis(2-hydroxyethyl)-p-phenylenediamine sulfate 0.0 to 5%.

The following are used as coupler:
Resorcinol 0.1 to 5%, 4-chlororesorcinol 0.1 to 5%, 2-methylresorcinol 0.1 to 1-naphthol 0.1 to 1%, m-aminophenol 0.1 to 5%, p-amino-o-cresol 0.1 to 5%, 5-amino-6-chloro-o-cresol 0.1 to 5%, 2-methyl-5-hydroxyethylaminophenol 0.1 to 5%, 2,4-diaminophenoxyethanol (HCl or H₂SO₄) 0.1 to 5%, 1,5-dihydroxynaphthalene 0.1 to 3%, 1,6 dihydroxynaphthalene 0.1 to 3%, 2,6-diaminopyridine 0.1 to 3%, 2-amino-4-hydroxyethylaminoanisole sulfate 0.1 to 5%, 2-amino-3-hydroxypyridine 0.1 to 5%.

The following are used as direct dye:
4-nitro-o-phenylenediamine 0.1 to 5%, 2-nitro-p-phenylenediamine 0.1 to 5%, 6-chloro-4-nitro-2-aminophenol 0.1 to 10%, sodium picramate 0.1 to 2%, picramic acid 0.1 to 2%, 4-amino-3-nitrophenol 0.1 to 4%, 4-hydroxypropylamino-3-nitrophenol 0.1 to 5%, 3-nitro-p-hydroxyethylaminophenol 0.1 to 5%, HC red 3 and 13, each 0.1 to 5%, HC yellow 2, 4 and 5, each 0.1 to 5%, HC blue 2 0.1 to 5%, basic red 2, 22 46, 51 and 76, each 0.1 to 4% basic blue 3, 7, 9, 26, 47 and 99, each 0.1 to 3%, basic yellow 11, 28 and 57, each 0.1 to 3%, basic brown 4, 16 and 17, each 0.1 to 3%, basic violet 4 and 14, each 0.1 to 3%, disperse violet 1 0.1 to 3%.

The following are used as active treatment substances:
Panthenol 0.1 to 5%, allantoin 0.1 to 0.5%, synthetic oils 1 to 5%, silicones 1 to 5%, vegetable oils such as jojoba oil, wheat-germ oil, corn oil, meadowfoam seed oil, each 1 to 10%, vitamins E—acetate 1 to 15%, UVA- and UVB filters 1 to 5%, silk proteins 1 to 4%, keratin hydrolysate 1 to 4%, collagen hydrolysate 1 to 4%, wheat protein 1 to 4%, elastin hydrolysate 1 to 4%.

The following are used as alkalizing agents:
Ammonium hydroxide 1 to 25%, ethanolamine 1 to 100%, aminomethylpropanol 1 to 100%, sodium hydroxide 1 to 10%, potassium hydroxide 1 to 10%.

The following is used as peroxide:
Hydrogen peroxide 1 to 35%.

The production of mixtures will be explained in detail using a few examples for a better understanding. The 11 bags based on a device provided with 11 bags in accordance with the previously described contents consequently contain the previously described filling contents. This concerns seven base components, two alkalizing agents and one bag with peroxide and one with pure carrier mass (see Table 1). It is of course just as possible to use another number of bags with other base components or another composition. The computer program and the recipes would then have to be correspondingly altered. The example with eleven bags is intended only to explain in detail the use of the base components for different tones and for the use as classic, ammonia-containing hair dye or as intensive toner without ammonia.

The following cream carrier mass 018 (without developer, coupler, direct dye) serves by way of example as a creamy carrier mass with treatment substances in which carrier mass the intermediates (developer and coupler) can be dissolved individually and in certain concentrations that can also differ if necessary:

| | |
|---|---|
| Oleic acid | 2.00% |
| Oleth-20 | 3.60% |
| Cetearyl alcohol | 15.00% |
| Sodium hydroxide 10% | 1.50% |
| EDTA | 0.10% |
| Sodium lauryl sulfate | 0.50% |
| Sodium sulfite | 0.40% |
| Ascorbic acid | 0.10% |
| Perfume | 0.30% |
| Hydrolyzed keratin | 0.50% |
| Aqua | qs |

Any desired color shadings can be composed with base components based on this carrier mass which can be obtained, depending on the type of coloring, with ammonia and/or other alkalizing agents. The examples cited in Table 1 show that classic hair dyes with ammonia or intensive toners can be produced in accordance with the described production method depending on the recipe and the alkalizing agents used (bags 9, 10).

TABLE 1

| | | Intensive Toners Without | | | | | |
|---|---|---|---|---|---|---|---|
| Bag- | | Ammonia | | | Hair Dyes with Ammonia | | |
| No. | Bag Contents | Blond | Violet | Red | Blond | Violet | Red |

Stage 1

| | | | | | | | |
|---|---|---|---|---|---|---|---|
| Bag 1 | p-Toluylendiamine 5% | 0.75 ml | 1.65 ml | 0.50 ml | 0.75 ml | 1.65 ml | 0.50 ml |
| | in Carrier Mass 018 | | | | | | |
| Bag 2 | p-Aminophenol 1% | 0.55 ml | 2.15 ml | 3.30 ml | 0.55 ml | 2.15 ml | 3.30 ml |
| | in Carrier Mass 018 | | | | | | |
| Bag 3 | p-Amino-o-Cresol 2% | — | 1.45 ml | 0.85 ml | — | 1.45 ml | 0.85 ml |
| | in Carrier Mass 018 | | | | | | |
| Bag 4 | Resorcin 2% | 0.60 ml | — | — | 0.60 ml | — | — |
| | in Carrier Mass 018 | | | | | | |
| Bag 5 | m-Aminophenol 1% | 0.15 ml | 0.45 ml | — | 0.15 ml | 0.45 ml | — |
| | in Carrier Mass 018 | | | | | | |
| Bag 6 | 1-Naphthol 1% | — | 0.50 ml | 2.00 ml | — | 0.50 ml | 2.00 ml |
| | in Carrier Mass 018 | | | | | | |
| Bag 7 | HC-Red #3 1% | — | — | 0.35 ml | — | — | 0.35 ml |
| | in Carrier Mass 018 | | | | | | |
| Bag 8 | Carrier Mass 018 | 4.50 ml | 0.80 ml | — | 4.50 ml | 0.80 ml | — |

Stage 2

| | | | | | | | |
|---|---|---|---|---|---|---|---|
| Bag 9 | Ethanolamine 10% | 3.00 ml | 3.00 ml | 3.00 ml | — | — | — |
| | in Carrier Mass 018 | | | | | | |
| Bag 10 | Ammonium hydroxide | — | — | — | 3.00 ml | 3.00 ml | 3.00 ml |
| | 6% | | | | | | |
| | in Carrier Mass 018 | | | | | | |

Stage 3

| | | | | | | | |
|---|---|---|---|---|---|---|---|
| Bag 11 | Cream Peroxide 6% | 10.00 ml | 10.00 ml | 10.00 ml | 10.00 ml | 10.00 ml | 10.00 ml |

| | | | | | | |
|---|---|---|---|---|---|---|
| Total read-to-use dye mass | 20.00 ml | 20.00 ml | 20.00 ml | 20.00 ml | 20.00 ml | 20.00 ml |

The examples in Table 1 show that not only 3 different color tones can be produced with 11 bags by altering the composition and/or amounts but also that these color tones are available both as classic, ammonia-containing hair dye as well as ammonia-free intensive toner in as far as the mixing of the bag contents 1 to 8 takes place in a precursor stage and only thereafter are the contents of bag 9 or 10 mixed in as alkalizing agent. Of course, as previously explained, more color tones can be produced by other compositions, in which case other recipes must be used. It is advantageous if the ratio of peroxide and base components is 50:50. However, it is absolutely possible to alter this ratio.

On the other hand, the sellers in the market divide the dyes into two separate areas—on the one hand the area of classic, ammonia-containing hair dyes, that simultaneously brighten the hair during the dyeing process and on the other hand intensive toners, that permit a tone-in-tone dying without brightening to be carried out. These two coloring systems are obligatorily produced and marketed individually. As a rule the classic hair dye assortment has approximately two to three times as many shades as the intensive toners have, partly because the space available to the hairdresser is limited.

According to this embodiment of the invention a hairdresser can now offer an almost unlimited assortment of classic, ammonia-free hair dyes as well as an intensive toning with only one to two additional bags (see Table 1). The described method is particularly advantageous during on-site production, that is, in the salon. It is thus associated not only with a significant reduction of storage space but also the great number of color tones is almost the same for both types of hair dyes.

As noted above, however, the prior art embodiment of machine 12 required a skilled operator to determine the appropriate formulation of dyes and chemicals and enter the formulation as a code into device 12. In accordance with another embodiment of the present invention, processes and apparatus are provided for enabling an untrained customer to operate machine 12 in any environment, for example a retail store, to mix, package and dispense a custom hair colorant.

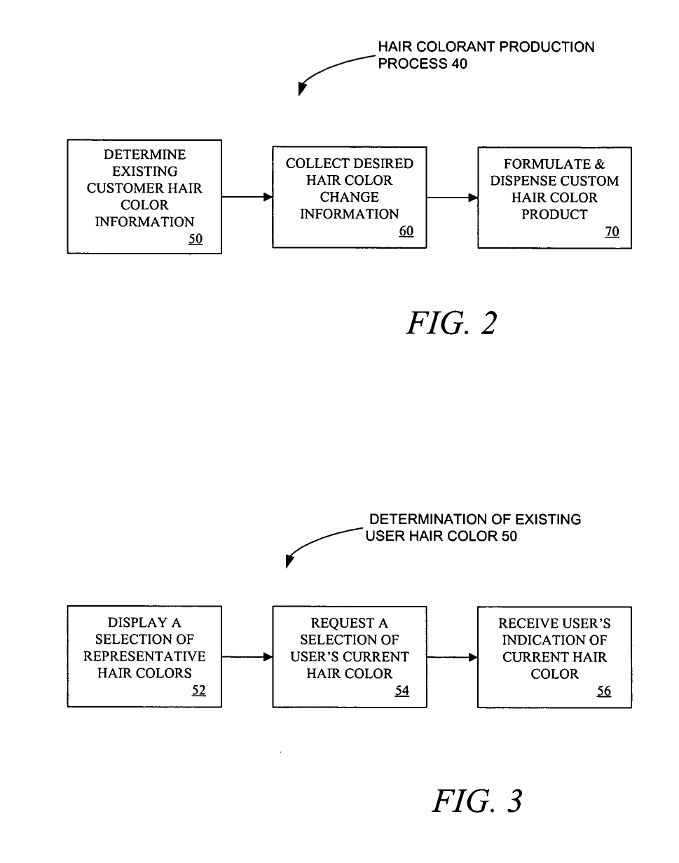

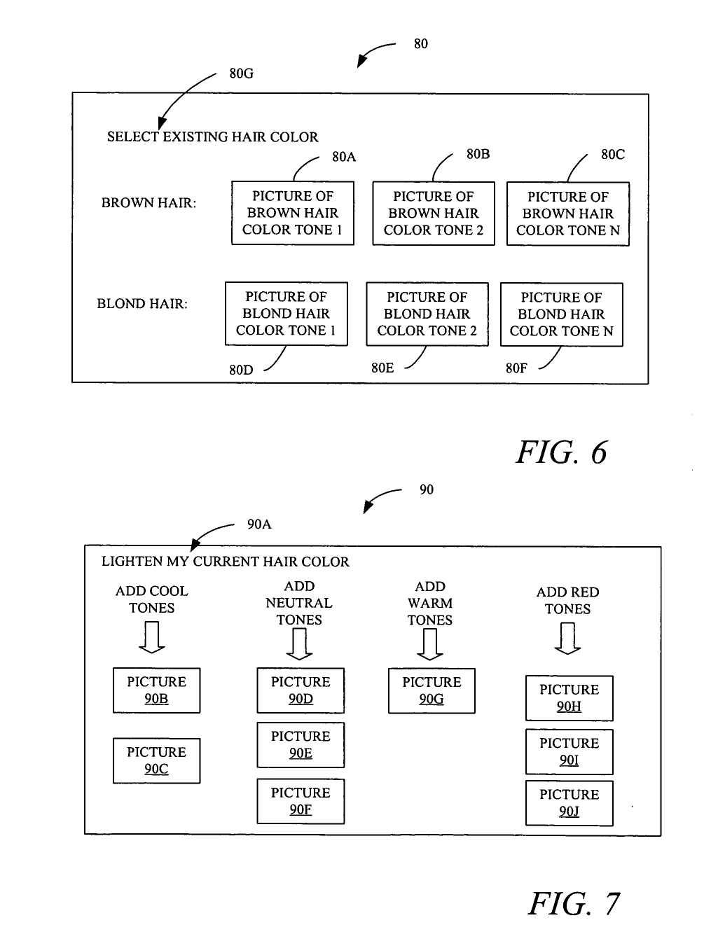

With reference to FIG. 2, a hair colorant production process 40 is shown cooperative with the graphical user interfaces of FIGS. 6 and 7 whereby device 12 can be operated by customer 30. Initially, the existing hair color of customer 30 is determined. With reference to FIGS. 3 and 6 together, this is done by displaying a selection of representative hair colors (step 52) on a graphical user interface screen 80 of display 28. As shown in FIG. 6, user interface 80 displays a variety of full color pictures of hair, each picture of varying color and tone. In the example shown, three pictorial samples of brown hair are displayed at 80A, B and C, while three pictorial samples of blond hair are displayed at 80D, E and F. An instruction 80G is provided directing the customer to select her existing hair color (step 54 of FIG. 3). The customer selection of their current, existing hair color is received (step 56) in one of many ways. For example, if display 28 includes a touch-screen, the user may simply touch the appropriate displayed color. Alternatively, the customer may enter their hair color information through a conventional keypad or keyboard. In an alternate embodiment of the invention, a camera may be used to actually sense the color of the customer's hair, either in lieu of or to validate another form of customer input.

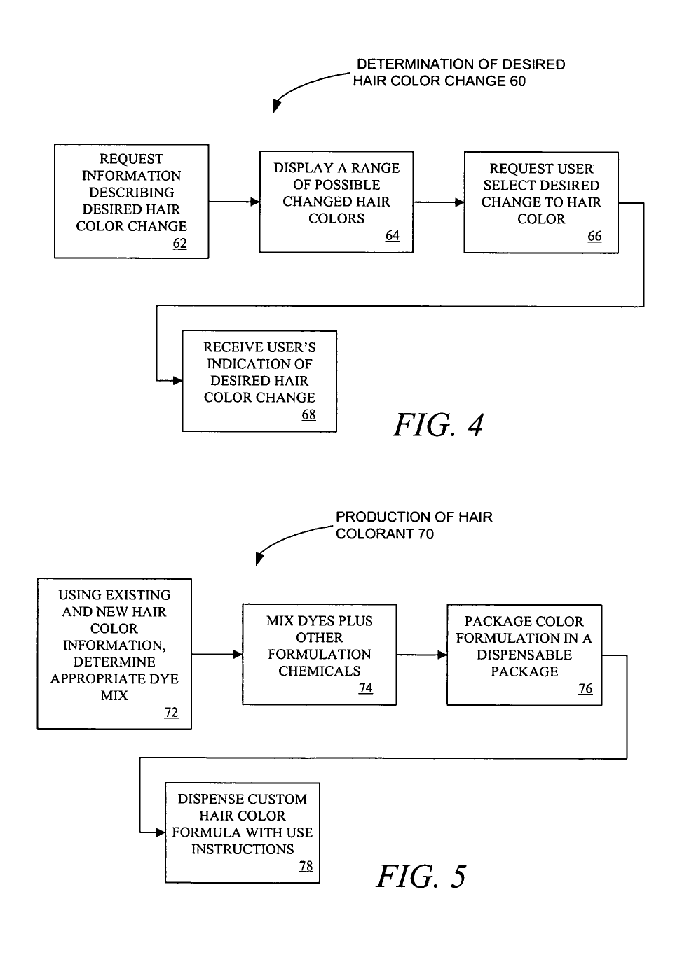

With reference back to FIG. 2, after determining customer 30's existing hair color, there is next determined her desired change to her existing hair color (step 60). With reference to FIGS. 4 and 7 together, there is displayed to customer 30 on display 28 a request for information regarding her desired hair color (step 62). This request may be, for example, a simple question such as: “What color would you like to dye your hair?” or “How would you like to change your hair color?” or some similar question. Appropriate questions may also be posed, the responses indicating whether the best hair color formulation for customer 30 would comprise a classic or intensive toner formulation of the types described above.

It is well known in the art that only certain color changes can be made to existing hair colors. For example, while it is possible to lighten dark brown hair, it is impossible to use hair color dyes to significantly darken dark brown hair. As another example, blond hair of an appropriate shade may be lightened or darkened. In accordance with the present invention, these well-known restrictions are stored in storage device 24 and used by controller 26 in communicating with customer 30. For example, guidance may be given as part of the question, for example informing the customer that based on her current hair color, it may only be possible to lighten, or darken her existing color. Input may be collected as noted above by touch-screen, keypad, keyboard or even voice recognition. Responsive to the customer's input of her desired hair color change, there is displayed a range of possible new hair colors that may be achieved through the use of dyes (step 64).

Considering FIG. 7, the display of available new hair colors may be in the form of a graphical user interface 90 including pictures on display 28. As shown, interface 90 indicates at 90A that customer 30 has chosen to lighten her current hair color. There are thus displayed a series of lighter hair colors achievable by: i) adding cool tones to the existing hair color, two of which are displayed at 90B, 90C, ii) adding neutral tones as shown in pictures 90D, E and F, iii) adding warm tones as shown in picture 90G and by adding red tones as shown in pictures 90H, I and J.

It will be understood that while, for purposes of illustration and explanation, relatively few choices of existing hair colors (FIG. 6) and desired changes (FIG. 7) have been shown, the entirety of existing hair colors and desired changes may be displayed in accordance with hair-coloring chemistry as described herein above. For example, more pictures may be shown on display 28 or multiple screens of pictures may be made available for display. Further, while the Figures diagrammatically illustrate a “PICTURE” it will be understood by the reader that an actual color picture is displayed to the customer on interface 28

With reference now back to FIG. 4, based on display 90, customer 30 is requested to indicate their desired hair color change (step 66) and that customer input is received (step 68) into controller 26 of machine 12.

With reference back to FIG. 2, based on the customer's indication of their existing hair color (step 50) and the desired change to that existing hair color (step 60), machine 12 is operated to formulate, mix, package and dispense a custom hair colorant (step 70). With reference now to FIG. 5, the process 70 for the production of hair colorant by machine 12 is shown whereby controller 26 uses the existing hair color and desired hair color change information collected from customer 30 in order to select and mix dyes and associated chemical components.

It will be understood by the reader that standardized processes for selecting and mixing hair color dyes of the type described above to achieve a particular result are well known to professionals in the art. Such processes are typically taught to professional hairdressers and hair colorists and require that they use materials containing actual charts showing ‘before’ and ‘after’ hair colors to select dye mixes. In accordance with the prior art, they then program the desired color results and dye combinations into a machine using codes as described with respect to German application 101 14 060 A1 in order to have the machine mix the appropriate dye formulation. Alternatively, if such a machine is not available, the colorist may simply mix the appropriate dyes from manually-dispensable containers.

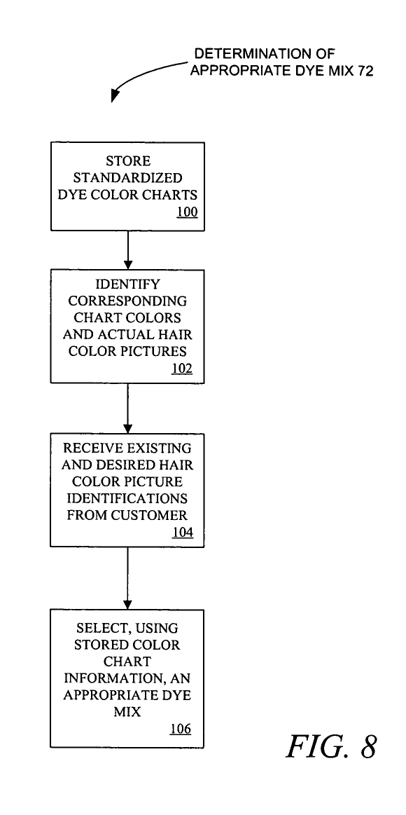

With reference to FIGS. 5 and 8 together, in accordance with the present invention, the data entered by customer 30 relating to existing hair color and the desired change is used to determine the appropriate dye mix (step 72). With reference to FIG. 8, the standardized dye color charts are programmed into machine 12 and stored in storage device 24 (step 100). The various entries on the charts are linked to the corresponding stored, displayable pictures of actual hair colors (step 102) of the type shown in FIGS. 6 and 7. As machine 12 receives the customer identification of an existing hair color and a desired change (step 103), controller 26 uses the identified pictures to access the stored color chart information in storage device 24 and select an appropriate dye mixture (step 106). It will thus be appreciated that, in accordance with the present invention, the appropriate dye mixture is obtained by customer 30's operation of machine 12 without the intervention or assistance of a trained colorist or operator.

With reference back to FIG. 5, the appropriate hair color dyes and associated chemical formulants are selected and mixed as described hereinabove (step 74). The custom hair-color mixture is then packaged (step 76) and dispensed to customer 30 (step 78).

There have thus been provided new and improved machines and processes that enable the formulation and dispensing of custom hair color products in real time by a machine capable of formulating essentially a limitless range of hair colorants. In one embodiment of the invention, systems and processes are provided which enable a customer to operate the machine through a simple user interface and without any training, intervention or assistance by a trained operator.

The invention enables the formulation of a broad range of hair colorants in real-time at salons and at non-traditional locations such as retail stores. Because the invention is capable of producing a vast number of hair color formulations from a relatively limited number of dyes and chemicals, it is capable of replacing large quantities of pre-packaged products. Operable by unskilled customers, the invention may be placed into many non-traditional locations such as retail stores. Because the selection of dyes and chemicals are programmed into the machine, human error is diminished and customers are likely to achieve their desired results. The present invention has application in the hair color industry, with particular application in the hair salon and retail store industries.

While the invention has been shown and described with respect to particular embodiments, it is not thus limited. Numerous changes, modifications, improvements and enhancements within the scope of the invention will now occur to those skilled in the art.

1. Apparatus for formulating a hair colorant, comprising:
a plurality of dyes;
means for receiving from a customer an indication of an existing hair color;
means for visually displaying to the customer a plurality of optional changes to the existing hair color;
means for receiving from the customer, responsive to the means for visually displaying, an indication of a desired change to the existing hair color; and
means for selecting from the plurality of dyes at least one selected dye for mixing into a hair colorant.

2. Apparatus in accordance with claim 1 wherein the means for receiving an indication of an existing hair color includes:
a display terminal for visually displaying a plurality of hair colors; and
means associated with the display terminal for receiving an indication of a selected one of the plurality of hair colors.

3. Apparatus in accordance with claim 2 wherein the means for receiving an indication of a selected one of the plurality of hair colors is a touch screen on which the plurality of hair colors are visually displayed.

4. Apparatus in accordance with claim 2 and further including means for mixing the at least one selected dye.

5. Apparatus in accordance with claim 4 and further including:
means for packaging the mixed at least one selected dye; and
means for dispensing the packaged dyes.

6. Apparatus in accordance with claim 2 wherein the means for selecting the at least one selected dye comprises a controller responsive to the indication of the existing hair color and the indication of the desired change to the existing hair color.

7. Apparatus in accordance with claim 6 and further comprising a storage device storing information corresponding to the existing hair color, information corresponding to the desired change to the existing hair color and information for selecting the at least one selected dye as a function of the existing hair color and the desired change to the existing hair color.

8. A method operable on a computer for formulating a hair colorant, comprising:
providing a plurality of dyes;
receiving into the computer from a customer an indication of an existing hair color;
visually displaying to the customer a plurality of optional changes to the existing hair color;
receiving into the computer from the customer, responsive to the visually displaying, an indication of a desired change to the existing hair color; and
selecting with the computer, from the plurality of dyes, at least one selected dye for mixing into a hair colorant.

9. A method in accordance with claim 8 wherein the receiving into the computer from a customer an indication of an existing hair color includes:
visually displaying on a display terminal a plurality of hair colors; and
receiving, based on the visually displaying, an indication of a selected one of the plurality of hair colors.

10. A method in accordance with claim 9 wherein the receiving an indication of a selected one of the plurality of hair colors includes providing a touch screen on which the plurality of colors are displayed.

11. A method in accordance with claim 9 and further including mixing the at least one selected dye.

12. A method in accordance with claim 11 and further including:
packaging the mixed at least one selected dye; and
dispensing the packaged dye.

13. A method in accordance with claim 8 wherein the selecting the at least one selected dye comprises operating a controller responsive to the indication of the existing hair color and the indication of the desired change to the existing hair color.

14. A method in accordance with claim 13 and further comprising storing information corresponding to the existing hair color, information corresponding to the desired change to the existing hair color and information for selecting the at least one selected dye as a function of the existing hair color and the desired change to the existing hair color.

15. A system for formulating a hair colorant, comprising:
a processor;
a memory connected to the processor and storing instructions to control the processor to perform the steps of:
identify a plurality of dyes;
receive from a customer an indication of an existing hair color;
visually display to the customer a plurality of optional changes to the existing hair color;
receive from the customer, responsive to the visually display, an indication of a desired change to the existing hair color; and
selecting, from the plurality of dyes, at least one selected dye for mixing into a hair colorant.

16. A system for formulating a hair colorant, comprising:
a processor;
a touch-screen display connected to the processor;
a memory connected to the processor and storing instructions to control the processor to perform the steps of:
display on the touch-screen display a plurality of hair colors;
receive from a customer through the touch-screen display an indication of an existing hair color;
display to the customer on the touch-screen display a plurality of optional changes to the existing hair color;
receive from the customer on the touch-screen display an indication of a desired change to the existing hair color; and
select, from a plurality of dyes, at least one selected dye for mixing into a hair colorant.

17. The system of claim 16 wherein the at least one selected dye is a plurality of selected dyes, and further comprising:
a mixer for mixing the plurality of selected dyes; and
a dispenser for dispensing the mixed plurality of dyes.

18. The system of claim 16 wherein the step of displaying to the customer on the touch-screen display a plurality of optional changes to the existing hair color comprises:
determining, based on the indication of the existing hair color, one or more possible changes to the existing hair color; and
displaying to the customer the one or more possible changes.

19. The system of claim 16 wherein the step of selecting, from a plurality of dyes, at least one selected dye for mixing into a hair colorant, includes the step of retrieving from the memory, based on the existing hair color and the desired change to the existing hair color an indicator of the selected dye.

20. The system of claim 17 and further including a packager for packaging the mixed plurality of dyes for use by the customer at a selected time and in a selected location.
